(19) United States
(12) Patent Application Publication
(10) Pub. No.: US 2016/0001036 A1
(43) Pub. Date: Jan. 7, 2016

(54) MEDICAL DRAINAGE TUBE OBSTRUCTION EXTRACTOR

(71) Applicant: Mayo Foundation for Medical Education and Research, Rochester, MN (US)

(72) Inventors: Terry P. Nickerson, Rochester, MN (US); Johnathon M. Aho, Rochester, MN (US); Matthew Schaff, Philadelphia, PA (US)

(21) Appl. No.: 14/770,636

(22) PCT Filed: Feb. 25, 2014
(86) PCT No.: PCT/US14/18232

§ 371 Date: Aug. 26, 2015

Related U.S. Application Data
(60) Provisional application No. 61/769,585, filed on Feb. 26, 2013.

(51) Int. Cl. A61M 25/00

(52) U.S. Cl. CPC A61M 25/00; A61M 25/0082; A61M 27/00

(57) ABSTRACT

Devices and methods can be used to extract obstructions from medical drainage tubes. For example, this document provides catheter-based devices for insertion in a drainage tube to enable irrigation of obstructions, aspiration, and mechanical removal of obstructions. In some examples, medical drainage tube devices are used to provide an avenue for the body to expel the byproducts of an infection or inflammation. Medical drainage tubes can create a pathway from a location in the interior of a body to the surface of the body for withdrawal of fluids and discharges from a wound, sore, cavity, and the like.

### CROSS-REFERENCE TO RELATED APPLICATIONS

This application claims the benefit of U.S. Provisional Application Ser. No. 61/769,585, filed Feb. 26, 2013. The disclosure of the prior application is considered part of (and is incorporated by reference in) the disclosure of this application.

### BACKGROUND

1. Technical Field

This document relates to devices and methods for extracting obstructions from medical drainage tubes. For example, this document relates to catheter-based devices for insertion in a medical drainage tube to facilitate irrigation, aspiration, and mechanical removal of drainage tube obstructions.

2. Background Information

In some examples, medical drainage tube devices are used to provide an avenue for the body to expel the byproducts of an infection or inflammation. Medical drainage tubes can create a pathway from a location in the interior of a body to the surface of the body for withdrawal of fluids and discharges from a wound, sore, cavity, and the like. Closed drainage is a system of tubing and other apparatus attached to the body to remove fluid in an airtight circuit that prevents environmental contaminants from entering the wound or cavity.

### SUMMARY

This document provides devices and methods for extracting obstructions from medical drainage tubes. For example, this document provides catheter-based devices for insertion in a medical drainage tube to enable irrigation of obstructions, aspiration, and mechanical removal of obstructions.

In general, one aspect of this document features a device for removing an obstruction from a medical drainage tube. The device comprises: a catheter with a proximal end and a tapered distal tip, the tapered distal tip including an aperture; an end cap fixedly coupled to the proximal end of the catheter, the end cap including a liquid sealing member; a wire with a first end and a second end, the wire being generally coaxial with the catheter and intersecting the liquid sealing member, wherein the first end is arranged to be manipulated by an operator of said device; and an expandable member at the second end of the wire, wherein the expandable member is arranged to expand from a low-profile configuration to an enlarged configuration.

The foregoing and other embodiments can each optionally include one or more of the following features, alone or in combination. The expandable member may be arranged to be in the low-profile configuration when the expandable member is located within the catheter, and the expandable member may be arranged to be in the enlarged configuration when the expandable member is outside of the catheter. The expandable member may be a brush having a plurality of flexible bristles. The device may further comprise a fitting attached to and in fluid communication with the catheter, wherein the fitting is configured to couple with an irrigation liquid source and to pass liquid into the catheter.

In general, another aspect of this document features a method for removing an obstruction from a medical drainage tube. The method comprises: inserting a device into a lumen of the medical drainage tube; passing the distal tip through the obstruction; causing the expandable member to emerge out from inside the catheter and through the aperture, whereby the expandable member reconfigures from the low-profile configuration to the enlarged configuration; and retracting the device from the medical drainage tube such that the expandable member contacts the obstruction to move the obstruction out of the medical drainage tube. The device comprises: a catheter with a proximal end and a distal tip, the distal tip including an aperture; an end cap fixedly coupled to the proximal end of the catheter; a wire with a first end and a second end, the wire being generally coaxial with the catheter, wherein the first end is arranged to be manipulated by an operator of the device; and an expandable member at the second end of the wire, wherein the expandable member is arranged to expand from a low-profile configuration to an enlarged configuration.

The foregoing and other embodiments can each optionally include one or more of the following features, alone or in combination. The method may further comprise injecting an irrigation liquid into the catheter. In addition, the method may further comprise suctioning the irrigation liquid into an aspiration catheter.

In another general aspect, this document features another device for removing an obstruction from a medical drainage tube. The device comprises: a catheter with a proximal end and a tapered distal tip, the tapered distal tip including an aperture; an end cap fixedly coupled to the proximal end of the catheter, the end cap including a liquid sealing member; an aspiration catheter with a first end portion and a second end portion; and an expandable member at the second end of the wire, wherein the expandable member is arranged to expand from a low-profile configuration to an enlarged configuration. In some embodiments, the aspiration catheter is generally coaxial with the catheter and intersecting the liquid sealing member. The first end portion of the aspiration catheter is arranged to be coupled to a suction source and manipulated by an operator of the device.

In another general aspect, this document features another method for removing an obstruction from a medical drainage tube. The method comprises: inserting a device into a lumen of the medical drainage tube; passing the distal tip through the obstruction; causing the expandable member to emerge out from inside the catheter and through the aperture, whereby the expandable member reconfigures from the low-profile configuration to the enlarged configuration; and retracting the device from the medical drainage tube such that the expandable member contacts the obstruction to move the obstruction out of the medical drainage tube. The device comprises: a catheter with a proximal end and a distal tip, the distal tip including an aperture; an end cap fixedly coupled to the proximal end of the catheter; an aspiration catheter with a first end portion and a second end portion; and an expandable member at the second end of the wire, wherein the first end is arranged to be manipulated by an operator of the device; and an expandable member at the second end of the wire, wherein the expandable member is arranged to expand from a low-profile configuration to an enlarged configuration. In some embodiments, the aspiration catheter is generally coaxial with the catheter and intersecting the liquid sealing member. The first end portion of the aspiration catheter is arranged to be coupled to a suction source and manipulated by an operator of the device.

In various implementations of the method, the method may further comprise injecting an irrigation liquid into the catheter. The method may also optionally include aspirating the irrigation liquid through one or more openings in the second end portion of the aspiration catheter.

Particular embodiments of the subject matter described in this document can be implemented to realize one or more of the following advantages. In some embodiments, the devices and methods provided herein for removing obstructions from drainage tubes can reduce or prevent patient fluid accumulation that may otherwise progress to hematoma or abscess formation. In some embodiments, the devices and methods provided herein can reduce or eliminate the need for surgical replacement of a clogged drainage tube. In some embodiments, the devices and methods provided herein can reduce the potential for secondary illnesses, reduce patient discomfort, and lower healthcare costs by ensuring drainage tube patency using an easy-to-use device and low risk procedure.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described herein. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description herein. Other features, objects, and advantages of the invention will be apparent from the description and drawings, and from the claims.

DESCRIPTION OF THE DRAWINGS

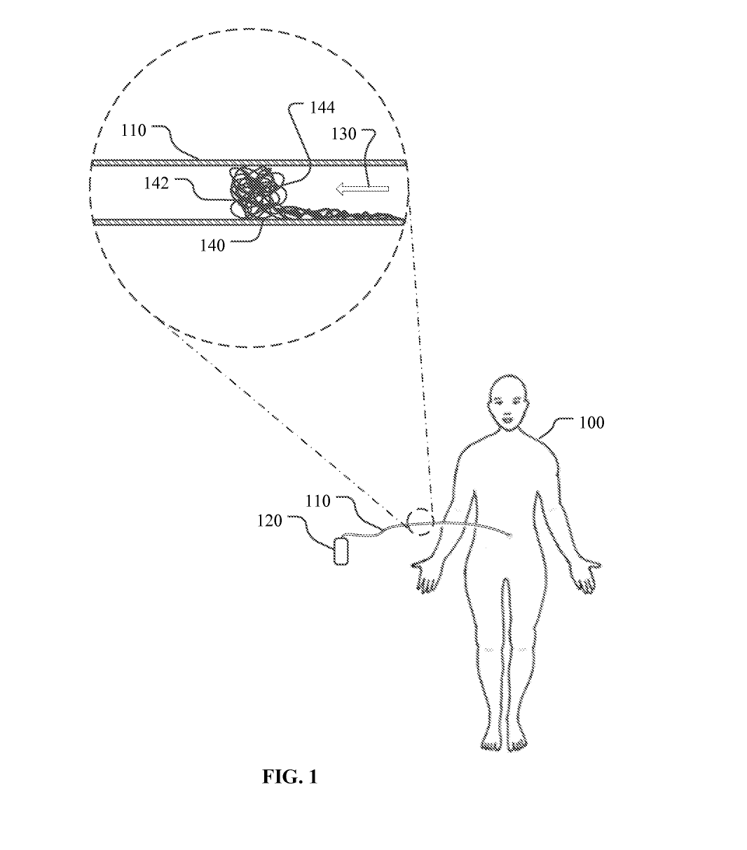

FIG. 1 is a schematic diagram of a surgical drainage tube system with an obstruction in the tube.

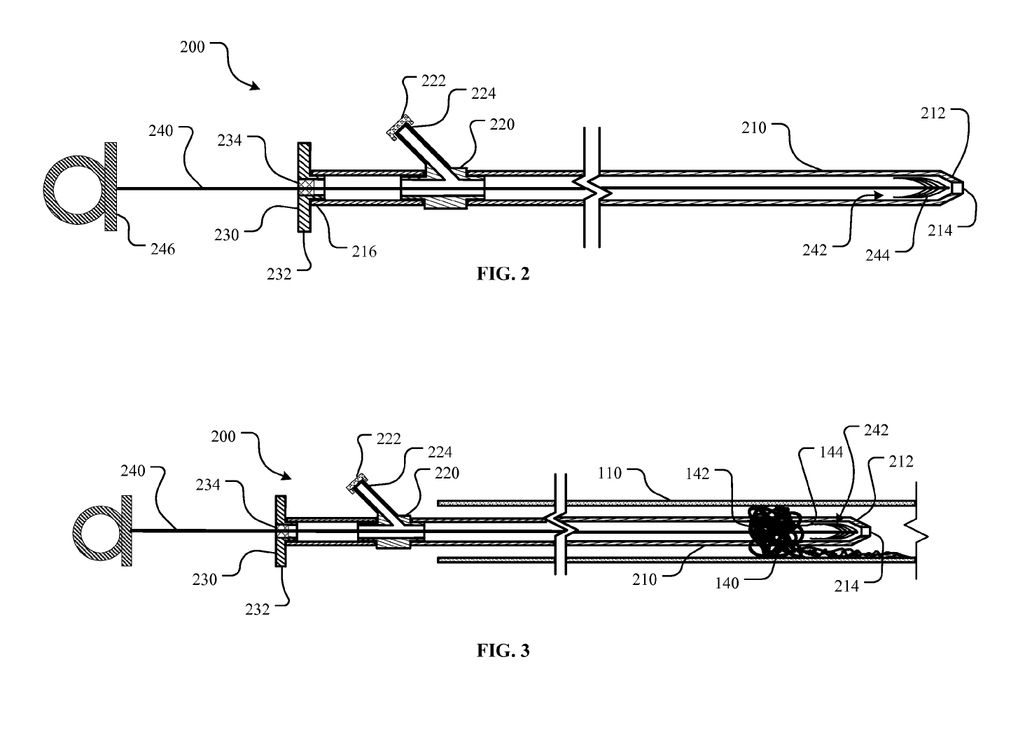

FIG. 2 is a longitudinal cross-sectional view of a drainage tube obstruction extractor device in accordance with some embodiments provided herein.

FIGS. 3-5 are a series of illustrations depicting a drainage tube obstruction extractor device removing an obstruction from a drainage tube in accordance with some embodiments provided herein.

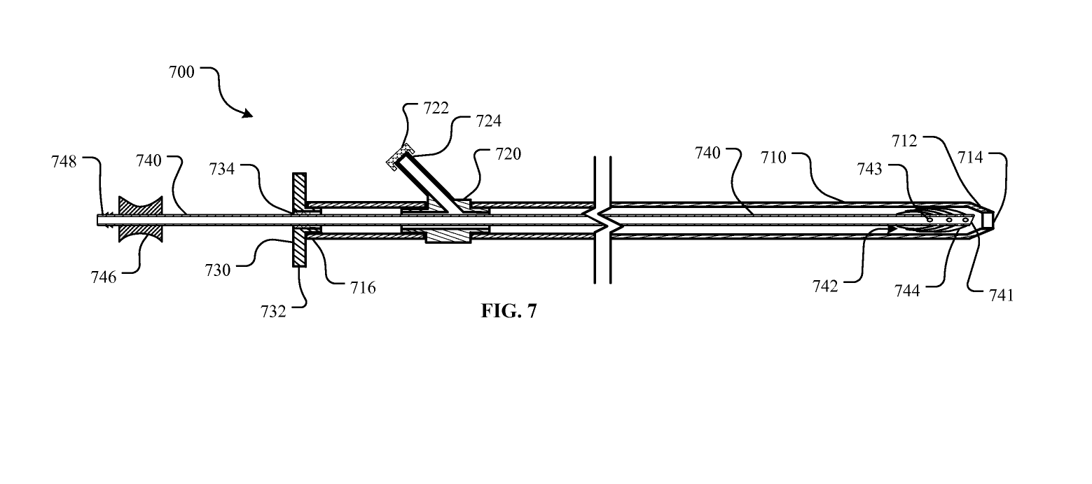

FIG. 6 is a flowchart of an exemplary method of extracting an obstruction from a medical drainage tube.

FIG. 7 is a longitudinal cross-sectional view of another drainage tube obstruction extractor device in accordance with some embodiments provided herein.

Like reference numbers represent corresponding parts throughout.

### DETAILED DESCRIPTION

Medical drainage tubes can become obstructed with blood clots and other debris, despite adequate drainage tube care such as stripping the drainage tube and increasing suction. This document provides catheter-based devices and methods for extracting obstructions from medical drainage tubes, by enabling both irrigation of obstructions, aspiration, and mechanical removal of obstructions. Using the devices and methods provided herein, some drainage tube obstructions can be removed without requiring replacement of the drainage tube by re-operation or the use of radiological guidance.

In some embodiments, the devices provided herein are catheters containing a wire that includes an expandable brush-like member at the distal end of the wire. The catheter can be inserted in a drainage tube, and the distal tip of the catheter can be passed through the drainage tube obstruction. In some embodiments, the distal tip of the catheter includes a tapered leading end to facilitate passage of the catheter's distal tip through the obstruction. In some instances, the obstruction can be softened using irrigation liquid injected through the devices provided herein. The irrigation liquid may loosen or dislodge the obstruction thereby facilitating its removal from the drainage tube.

In some embodiments, when the distal tip of the catheter has passed through the obstruction, the catheter can be pulled-back in a proximal direction while maintaining the wire substantially stationary. The relative movement between the catheter and the wire can cause the distal end of the wire to protrude from the tip of the catheter. In some embodiments, the brush-like member on the distal end of the wire will emerge out of the catheter, and bristles of the brush-like member will expand. In some embodiments, the catheter and wire can then be retracted from the drainage tube, and the bristles of the brush-like member will mechanically force the obstruction out of the drainage tube.

With reference to FIG. 1, a patient 100 is depicted as connected to a medical drainage tube 110 that transmits bodily fluids to a collection bag 120. In the magnified view, an arrow 130 depicts the intended direction of flow of the bodily fluids through the tube 110.

An obstruction 140 is shown within tube 110. In some instances, obstruction 140 may be a clot (e.g., a blood clot, or a clot of other bodily fluids). In some instances, obstruction 140 may be a collection of debris. In some instances, obstruction 140 may be another type of material. Obstruction has a proximal side 142 and a distal side 144.

Obstruction 140 may partially or fully block the flow of fluids through the drainage tube 110. In such cases, fluids may not be expelled from patient 100 as desired, and patient's 100 health or healing rate may be negatively impacted. Therefore, removal of obstruction 140 from drainage tube 110 is desirable.

With reference to FIG. 2, an exemplary drainage tube obstruction extractor 200 in accordance with some embodiments is illustrated. Extractor 200 is shown in a longitudinal cross-sectional view to promote good visualization and understanding of its components. Some components of example extractor 200 include a catheter 210, a luer fitting connector 220, an end cap 230, and a wire 240.

In some embodiments, catheter 210 is a polymeric tube that is circular in cross-section. Catheter 210 has at least one lumen. In some embodiments, catheter 210 is an extruded tube made from plastic material such as vinyl, nylon, polyurethane, polyethylene terephthalate, or silicone to provide a few examples. In some embodiments, catheter 210 is wire-reinforced. In some embodiments, catheter 210 has a conically tapered distal tip 212. In some embodiments, distal tip 212 is a thermo-formed end of catheter 210. For example, heat or RF energy can be applied to the end of catheter 210, and distal tip 212 can be formed in a die to attain the tapered shape. In some embodiments, distal tip 212 is a separate component that is attached to the end of catheter 210 during an assembly process. For example, distal tip 212 can be a separate molded part that is coupled onto the end of catheter 210.

Distal tip 212 includes an aperture 214. In some embodiments, aperture 214 enables the distal end of wire 240 to emerge from catheter 210. In some embodiments, aperture 214 is used to allow irrigation liquid to be emitted from catheter 210. In some embodiments, aperture 214 enables both emergence of wire 240 and transmission of irrigation liquid. In some embodiments, more than one aperture 214 is included at distal tip 212. For example, in some such embodiments, additional radial holes are included to provide additional irrigation liquid transmission ports.

A proximal end 216 of catheter 210 is located opposite of distal tip 212. Proximal end 216 can be attached to end cap 230. In some embodiments, proximal end 216 is attached to end cap 230 by an adhesive, solvent bonding, a barbed connection, a compression clamp (or a combination of such methods) to provide a few examples.

End cap 230 can be a polymeric or metallic fitting. For example, in some embodiments, end cap 230 is a molded plastic fitting. In some embodiments, end cap 230 is molded of polycarbonate, polystyrene, or polyvinyl chloride (PVC), for example. In some embodiments, end cap 230 includes flange 232. Flange 232 can provide a convenient place for a clinician operator to grasp onto extractor 200. End cap 230 can also include a gel lock 234. In some embodiments, gel lock 234 is a sealing material that can allow wire 240 to pass through end cap 230 while maintaining a liquid seal. In some embodiments, gel lock 234 can be a urethane gel, silicone gel, polymer hydrogel, or other suitable flexible lubricous material or combination of materials. In some embodiments, gel lock 234 can also comprise a flexible fabric or membranous material, or such materials can be combined with the gel materials.

Example drainage tube obstruction extractor 200 can include luer fitting connector 220. Luer fitting connector 220 can be in fluid communication with catheter 210. Luer fitting connector 220 can be used to inject irrigation liquid into catheter 210 as desired. In some embodiments, luer fitting connector 220 can be incorporated with end cap 230, such that the two are a unitary component. In some embodiments, luer fitting connector 220 can be a separate component that is included in-line with catheter 210. For example, luer fitting connector 220 can be a molded plastic component that is attached to catheter 220 by an adhesive, solvent bonding, a barbed connection, a compression clamp (or a combination of methods) to provide a few examples. Luer fitting connector 220 includes a cap 222 and a barrel 224. To add irrigation liquid to catheter 210, cap 222 can be removed and a compatible syringe or tube can be coupled to barrel 224. In some embodiments, barrel 224 is a standard male or female luer lock fitting. In some embodiments, other types of connection features, such as a barbed fitting, can be included on barrel 224.

Wire 240 is generally coaxial with catheter 210. Wire 240 intersects with and passes through gel lock 234. Wire 240 can be made of metallic or polymeric materials, or a combination thereof. In some embodiments, wire 240 is made of aluminum, stainless steel, steel alloys, nitinol, titanium, titanium alloys, nylon, polyester, polyethylene, polypropylene, polystyrene, PTFE, carbon fiber, or PVC to name a few examples. In some embodiments, wire 240 is a single strand. In some embodiments, wire 240 has two or more strands that are twisted with each other or otherwise combined together to form a single elongate member.

Example drainage tube obstruction extractor 200 includes a handle 246 at the proximal end of wire 240. In some embodiments, no handle 246 is included. In some embodiments, other types of handles are included.

A brush-like member 242 is at the distal end of wire 240. In some embodiments, other types of devices are used at the distal end of wire 240. For example, in some embodiments, rather than brush-like member 242 or in combination with brush-like member 242, the distal end of wire 240 can include a corkscrew member, inflatable member, grasping device, cutting device, and the like.

Brush-like member 242 includes bristles 244. Bristles 244 can be compressed in a low-profile configuration inside catheter 210. As described further herein, bristles 244 can expand when brush-like member 242 emerges from aperture 214 of catheter 210.

Bristles 244 can be made from a variety of materials including polymeric and metallic materials. In some embodiments, bristles 244 are made from dissolvable or biosorbable materials. In some embodiments, bristles 244 are nylon, polyester, polyethylene, polypropylene, polystyrene, PTFE, carbon fiber, or PVC to name a few polymeric examples. In some embodiments, bristles are aluminum, stainless steel, stainless steel alloys, titanium, titanium alloys, nitinol, or nitinol alloys to name a few metallic examples. In some embodiments, a combination of materials are used for bristles 244. In some embodiments, all individual bristles 244 are substantially a uniform length. In some embodiments, brush-like member 242 includes individual bristles 244 that have a variety of lengths.

In some embodiments, bristles 244 are attached to wire 240 by being compressed between wound wire filament strands that comprise wire 240. In some embodiments, bristles 244 are flocked to wire 240, using for example, an adhesive. In some embodiments, brush-like member 242 is a separate component that is attachable on the end of wire 240, using a threaded coupling, welding, by adhesive, and/or other suitable methods.

The length of example drainage tube obstruction extractor 200 can differ in various embodiments. For example, catheter 210 can range, in some embodiments, from about 20 cm to about 60 cm, about 50 cm to about 90 cm, about 80 cm to about 120 cm or longer. Wire 240 can range, in some embodiments, from about 30 cm to about 70 cm, about 60 cm to about 100 cm, about 90 cm to about 130 cm or longer. In general, wire 240 can be longer than catheter 210 so brush-like member 242 can be arranged to protrude from distal tip 212 of catheter 210.

With reference to FIG. 3, example drainage tube obstruction extractor 200 is shown inserted in medical drainage tube 110 to remove obstruction 140. The illustration is a longitudinal cross-sectional view to enhance visualization and understanding of the devices and methods provided herein. In the illustrated configuration, collection bag 120 (refer to FIG. 1) has been uncoupled from tube 110 to allow access to the interior of tube 110.

A clinician will insert extractor 200 in tube 110, and advance extractor 200 so that distal tip 212 will approach obstruction 140. In some instances, the tapered profile of distal tip 212 will enable the clinician to push distal tip 212 through obstruction 140 as depicted. In some instances, penetration of obstruction 140 by distal tip 212 may be difficult, and irrigation liquid can be delivered to soften, deteriorate, and/or dislodge obstruction 140. For example, in some cases an irrigation liquid of sterile 0.9% normal saline, sterile water, or other suitable liquids can be used.

To deliver irrigation liquid, the clinician can first position aperture 214 close to proximal side 142 of obstruction 140. A source of irrigation liquid can be attached to luer fitting connector 220. Irrigation liquid can be injected via luer fitting connector 220 into the interior of catheter 210. Gel lock 234 will prevent irrigation liquid from flowing out end cap 230—causing flow of irrigation liquid out of catheter 210 through aperture 214 at distal tip 212. Irrigation liquid will thereby come into contact with obstruction 140 to soften, deteriorate, and/or dislodge obstruction 140. Such softening of obstruction 140 can facilitate penetration of obstruction 140 by distal tip 212, to attain the configuration shown where distal tip is located beyond the distal side 144 of obstruction 140. At this stage, brush-like member 242 is located within catheter 210 in a low-profile configuration.

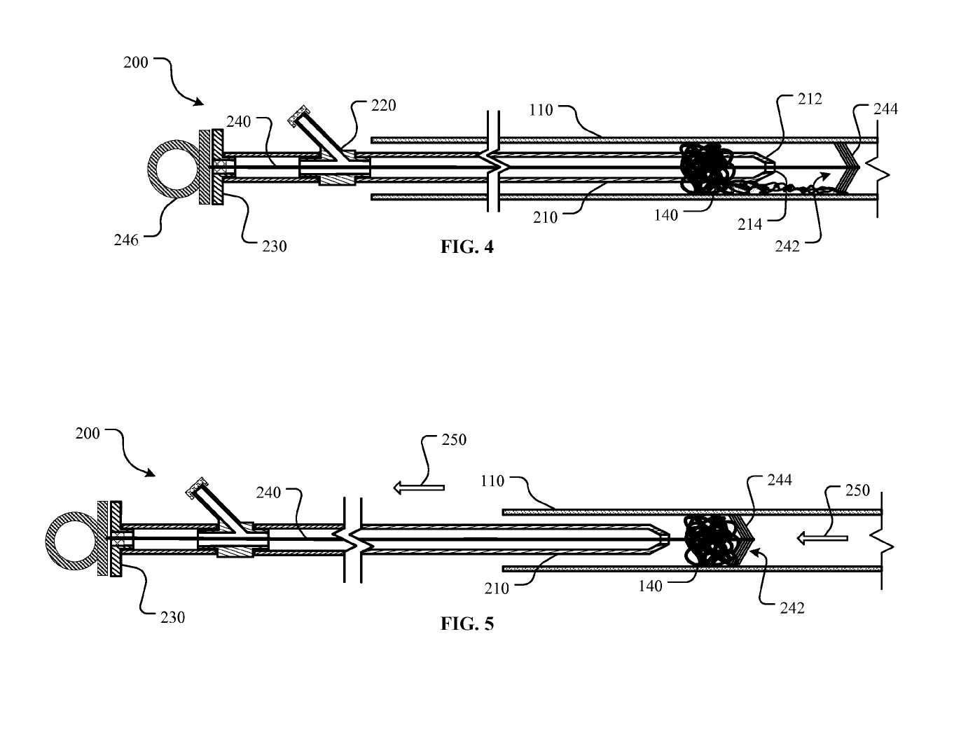

With reference to FIG. 4, wire 240 has been pushed distally in relation to catheter 210 such that brush-like member 242 has emerged from distal tip 212. Brush-like member 242 is positioned on the distal side of obstruction 140.

At this stage, bristles 244 have expanded towards their natural orientation in an enlarged configuration (whereas bristles 244 were previously constrained within catheter 210). In some embodiments, bristles 244 may expand to make contact with the inner wall of catheter 210. In some embodiments, some individual bristles 244 may make contact with the inner wall of catheter 210 while other individual bristles 244 do not make contact. In some embodiments, no bristles 244 make contact with the inner wall of catheter 210.

In some implementations, rather than pushing wire 240 distally, a clinician may deploy brush-like member 242 by pulling back catheter 210 while maintaining the position of wire 240 substantially stationary. In some embodiments, a combination of techniques is used to deploy brush-like member 242. In any case, brush-like member 242 is caused to emerge from aperture 214 of catheter 210 such that brush-like member 242 is positioned distally of obstruction 140.

In some cases, the clinician may choose to inject irrigation liquid at this stage. The addition of irrigation liquid may loosen obstruction 140 to facilitate removal of obstruction 140. Irrigation liquid can be injected into catheter 210 via luer fitting connector 220 as described herein.

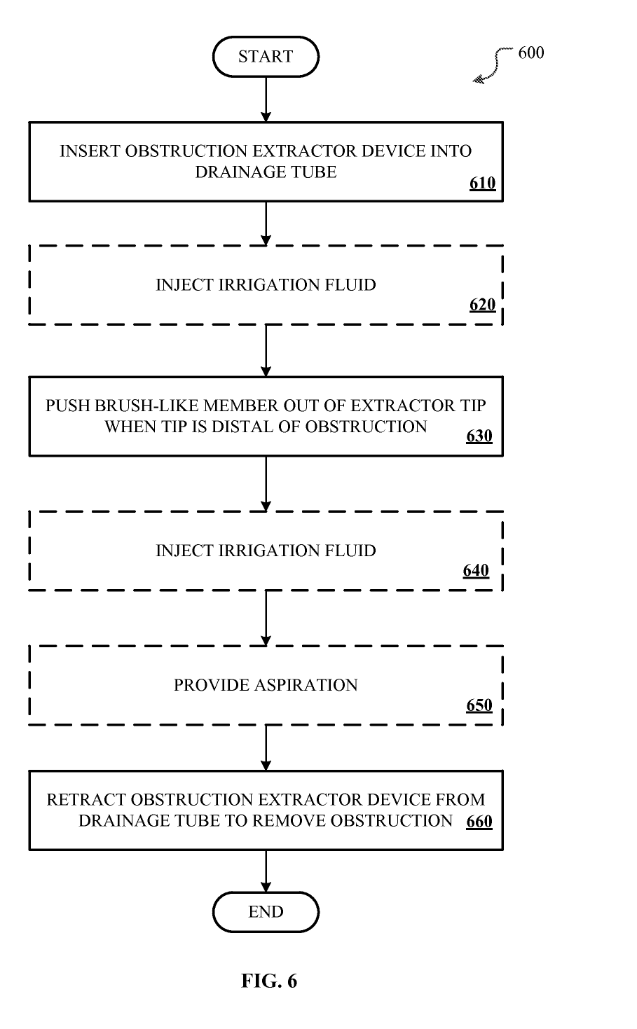

With reference to FIG. 5, example drainage tube obstruction extractor 200 is shown being pulled proximally in relation to medical drainage tube 110 to remove obstruction 140. Arrows 250 represent the relative direction of movement of extractor 200 in relation to tube 110.

As the clinician pulls extractor 200 proximally, bristles 244 contact obstruction 140. As extractor 200 is pulled further proximally, bristles 244 push obstruction 140 proximally in relation to tube 110. Irrigation liquid can be added as desired. In this manner, obstruction 140 can be pulled out of tube 110.

After removal of obstruction 140 from tube 110, collection bag 120 can be re-coupled to tube 110. At this stage, drainage tube 110 is free from obstruction 140 and the proper function of drainage tube 110 has been restored.

FIG. 6 is a flowchart of an exemplary process 600 for removing an obstruction from a medical drainage tube using the catheter-based drainage tube obstruction extractor devices provided herein. As described herein, in general the obstruction extractor devices provided herein include a catheter with a distal tip, a wire within the catheter, and a brush-like member at the distal end of the wire.

At operation 610, the extractor device is inserted in a drainage tube that contains an obstruction. The extractor device is advanced within the drainage tube so that the distal tip approaches the obstruction. At operation 620, irrigation liquid is optionally injected via the extractor device to soften the obstruction. The distal tip of the extractor device is advanced through the obstruction such that the distal tip of the extractor device is positioned beyond the distal side of the obstruction.

At operation 630, the extractor device's brush-like member is pushed out of the distal tip of the extractor device's catheter. The brush-like member has bristles that reconfigure from a low-profile arrangement (when in the catheter) to an expanded configuration as the brush-like member emerges from the catheter. At operation 640, irrigation liquid can optionally be injected to soften/loosen the obstruction.

At operation 650, at least some of the irrigation liquid can be optionally aspirated by adding suction in the region near the obstruction using an aspiration catheter (e.g., refer to FIG. 7). In addition, some material from the obstruction may be suctioned through the aspiration catheter.

At operation 660, the extractor device is retracted from within the drainage tube. The bristles of the brush-like member make contact with the obstruction. The bristles push the obstruction to move it within the drainage tube as the extractor device is retracted. The obstruction is removed from the drainage tube as the extractor device is fully removed from the drainage tube.

Referring now to FIG. 7, another example embodiment of a drainage tube obstruction extractor 700 in accordance with some embodiments is illustrated. Extractor 700 is shown in a longitudinal cross-sectional view to promote good visualization and understanding of its components. Some components of example extractor 700 include a catheter 710, a luer fitting connector 720, an end cap 730, and an aspiration catheter 740. Aspiration catheter 740 is provided in this embodiment as a source of suction for removing irrigation fluid and materials from clots and other obstructions.

In some embodiments, catheter 710 is a polymeric tube that is circular in cross-section. Catheter 710 has at least one lumen. In some embodiments, catheter 710 is an extruded tube made from plastic material such as vinyl, nylon, polyurethane, polyethylene terephthalate, or silicone to provide a few examples. In some embodiments, catheter 710 is wire-reinforced. In some embodiments, catheter 710 has a conically tapered distal tip 712. In some embodiments, distal tip 712 is a thermo-formed end of catheter 710. For example, thermo or RF energy can be applied to the end of catheter 710, and distal tip 712 can be formed in a die to attain the tapered shape. In some embodiments, distal tip 712 is a separate component that is attached to the end of catheter 710 during an assembly process. For example, distal tip 712 can be a separate molded part that is coupled onto the end of catheter 710.

Distal tip 712 includes an aperture 714. In some embodiments, aperture 714 enables the distal end of aspiration catheter 740 to emerge from catheter 710. In some embodiments, aperture 714 is also used to allow irrigation liquid to be emitted from catheter 710. In some embodiments, aperture 714 enables both emergence of wire 740 and transmission of irrigation liquid. In some embodiments, more than one aperture 714 is included at distal tip 712. For example, in some such embodiments, additional radial holes are included to provide additional irrigation liquid transmission ports.

A proximal end 716 of catheter 710 is located opposite of distal tip 712. Proximal end 716 can be attached to end cap 730. In some embodiments, proximal end 716 is attached to end cap 730 by an adhesive, solvent bonding, a barbed connection, a compression clamp (or a combination of such methods) to provide a few examples.

End cap 730 can be a polymeric or metallic fitting. For example, in some embodiments, end cap 730 is a molded plastic fitting. In some embodiments, end cap 730 is molded of polycarbonate, polystyrene, or polyvinyl chloride (PVC), for example. In some embodiments, end cap 730 includes flange 732. Flange 732 can provide a convenient place for a clinician operator to grasp onto extractor 700. End cap 730 can also include a gel lock 734. In some embodiments, gel lock 734 is a sealing material that can allow aspiration catheter 740 to pass through end cap 730 while maintaining a liquid seal. In some embodiments, gel lock 734 can be a urethane gel, silicone gel, polymer hydrogel, or other suitable flexible lubricous material or combination of materials. In some embodiments, gel lock 734 can also comprise a flexible fabric or membranous material, or such materials can be combined with the gel materials.

Example drainage tube obstruction extractor 700 can include luer fitting connector 720. Luer fitting connector 720 can be in fluid communication with catheter 710. Luer fitting connector 720 can be used to inject irrigation liquid into catheter 710 as desired. In some embodiments, luer fitting connector 720 can be incorporated with end cap 730, such that the two are a unitary component. In some embodiments, luer fitting connector 720 can be a separate component that is included in-line with catheter 710. For example, luer fitting connector 720 can be a molded plastic component that is attached to catheter 720 by an adhesive, solvent bonding, a barbed connection, a compression clamp (or a combination of methods) to provide a few examples.

Luer fitting connector 720 includes a cap 722 and a barrel 724. To add irrigation liquid to catheter 710, cap 722 can be removed and a compatible syringe or tube can be coupled to barrel 724. In some embodiments, barrel 724 is a standard male or female luer lock fitting. In some embodiments, other types of connection features, such as a barbed fitting, can be included on barrel 724.

Aspiration catheter 740 is generally coaxial with catheter 710. Aspiration catheter 740 intersects with and passes through gel lock 734. Aspiration catheter 740 can be made of metallic or polymeric materials, or a combination thereof In some embodiments, aspiration catheter 740 is made of aluminum, stainless steel, steel alloys, nitinol, titanium, titanium alloys, nylon, polyester, polyethylene, polypropylene, polystyrene, PTFE, carbon fiber, or PVC to name a few examples.

Aspiration catheter 740 defines at least one lumen, and in some embodiments, includes a proximal fitting 748, a distal end opening 741, and one or more distal end portion fenestrations 743. Proximal fitting 748 is configured for coupling to a suction source. For example, in some embodiments, proximal fitting 748 is a barbed fitting that can be readily connected to a suction tube.

When a suction source is connected to proximal fitting 748, a suction force (vacuum) is conveyed through the lumen of aspiration catheter 740 to distal end opening 741 and distal end portion fenestrations 743. Accordingly, liquids and other materials can be thereby drawn into the lumen of aspiration catheter 740 through distal end opening 741 and distal end portion fenestrations 743.

In some embodiments, aspiration catheter 740 includes a finger grip 746 at the proximal end portion of aspiration catheter 740. In some embodiments, no finger grip 746 is included. In some embodiments, other types of gripping adapters are included.

A brush-like member 742 is at the distal end of aspiration catheter 740. In some embodiments, other types of devices are used at the distal end of aspiration catheter 740. For example, in some embodiments, rather than brush-like member 742 or in combination with brush-like member 742, the distal end of aspiration catheter 740 can include a corkscrew member, inflatable member, grasping device, cutting device, and the like.

Brush-like member 742 includes bristles 744. Bristles 744 can be compressed in a low-profile configuration inside catheter 710. As described above in regard to bristles 244, bristles 744 can expand when brush-like member 742 emerges from aperture 714 of catheter 710.

Bristles 744 can be made from a variety of materials including polymeric and metallic materials. In some embodiments, bristles 744 are made from dissolvable or biosorbable materials. In some embodiments, bristles 744 are nylon, polyester, polyethylene, polypropylene, polystyrene, PTFE, carbon fiber, or PVC to name a few polymeric examples. In some embodiments, bristles are aluminum, stainless steel, stainless steel alloys, titanium, titanium alloys, nitinol, or nitinol alloys to name a few metallic examples. In some embodiments, a combination of materials are used for bristles 744. In some embodiments, all individual bristles 744 are substantially a uniform length. In some embodiments, brush-like member 742 includes individual bristles 744 that have a variety of lengths.

In some embodiments, bristles 744 are attached to aspiration catheter 740 by being flocked to aspiration catheter 740, using for example, an adhesive. In some embodiments, brush-like member 742 is a separate component that is attachable on the end of aspiration catheter 740, using a threaded coupling, welding, by adhesive, and/or other suitable methods.

The length of example drainage tube obstruction extractor 700 can differ in various embodiments. For example, catheter 710 can range, in some embodiments, from about 20 cm to about 60 cm, about 50 cm to about 90 cm, about 80 cm to about 120 cm or longer. Aspiration catheter 740 can range, in some embodiments, from about 30 cm to about 70 cm, about 60 cm to about 100 cm, about 90 cm to about 130 cm or longer. In general, aspiration catheter 740 can be longer than catheter 710 so brush-like member 742 can be arranged to protrude from distal tip 712 of catheter 710.

The technique for using example drainage tube obstruction extractor 700 can be substantially similar to the technique for using drainage tube obstruction extractor 200 as described above, except that drainage tube obstruction extractor 700 can also provide aspiration via aspiration catheter 740. The capability of drainage tube obstruction extractor 700 to provide aspiration can be beneficial in some circumstances. For example, when brush-like member 742 is protruding from distal tip 712 of catheter 710 and irrigation fluid is flowing through catheter 710, aspiration catheter 740 can suction up the irrigation fluid so as to allow for more irrigation to take place as compared to when no aspiration is being used. In another example, aspiration via distal end opening 741 and distal end portion fenestrations 743 can facilitate the removal of portions of an obstruction as the irrigation fluid breaks up the obstruction. Hence, aspiration can be used to improve the technique of clearing the obstruction out of drainage tubes.

In some embodiments, an additional suction pressure release feature can be added to aspiration catheter 740. This feature can enhance the ability of aspiration catheter 740 to provide suction even in the event that distal end opening 741 and/or distal end portion fenestrations 743 become occluded by materials.

While this specification contains many specific implementation details, these should not be construed as limitations on the scope of any invention or of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described herein as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described herein should not be understood as requiring such separation in all embodiments, and it should be understood that the described components and systems can generally be integrated together in a single product or packaged into multiple products.

Particular embodiments of the subject matter have been described. Other embodiments are within the scope of the following claims.

What is claimed is:

1. A device for removing an obstruction from a medical drainage tube, the device comprising:
a catheter with a proximal end and a tapered distal tip, said tapered distal tip including an aperture;
an end cap fixedly coupled to said proximal end of said catheter, said end cap including a liquid sealing member;
a wire with a first end and a second end, said wire being generally coaxial with said catheter and intersecting said liquid sealing member, wherein said first end is arranged to be manipulated by an operator of said device; and
an expandable member at said second end of said wire, wherein said expandable member is arranged to expand from a low-profile configuration to an enlarged configuration.

2. The device of claim 1, wherein said expandable member is arranged to be in said low-profile configuration when said expandable member is located within said catheter, and wherein said expandable member is arranged to be in said enlarged configuration when said expandable member is outside of said catheter.

3. The device of claim 1, wherein said expandable member comprises a brush having a plurality of flexible bristles.

4. The device of claim 1, further comprising a fitting attached to and in fluid communication with said catheter, wherein said fitting is configured to couple with an irrigation liquid source and to pass liquid into said catheter.

5. A method for removing an obstruction from a medical drainage tube, wherein said method comprises:
inserting a device into a lumen of said medical drainage tube, wherein said device comprises:
a catheter with a proximal end and a distal tip, said distal tip including an aperture;
an end cap fixedly coupled to said proximal end of said catheter;
a wire with a first end and a second end, said wire being generally coaxial with said catheter, wherein said first end is arranged to be manipulated by an operator of said device; and
an expandable member at said second end of said wire, wherein said expandable member is arranged to expand from a low-profile configuration to an enlarged configuration;
passing said distal tip through said obstruction;
causing said expandable member to emerge out from inside said catheter and through said aperture, whereby said expandable member reconfigures from said low-profile configuration to said enlarged configuration; and
retracting said device from said medical drainage tube such that said expandable member contacts said obstruction to move said obstruction out of said medical drainage tube.

6. The method of claim 5, further comprising injecting an irrigation liquid into said catheter.

7. A device for removing an obstruction from a medical drainage tube, the device comprising:
a catheter with a proximal end portion and a tapered distal tip portion, said tapered distal tip portion including an aperture;
an end cap fixedly coupled to said proximal end portion of said catheter, said end cap including a liquid sealing member;
an aspiration catheter with a first end portion and a second end portion, said aspiration catheter being generally coaxial with said catheter and intersecting said liquid sealing member, wherein said first end portion is arranged to be coupled to a suction source and manipulated by an operator of said device; and
an expandable member at said second end portion of said aspiration catheter, wherein said expandable member is arranged to expand from a low-profile configuration to an enlarged configuration.

8. The device of claim 7, wherein said expandable member is arranged to be in said low-profile configuration when said expandable member is located within said catheter, and wherein said expandable member is arranged to be in said enlarged configuration when said expandable member is outside of said catheter.

9. The device of claim 7, wherein said expandable member comprises a brush having a plurality of flexible bristles.

10. The device of claim 7, further comprising a fitting attached to and in fluid communication with said catheter, wherein said fitting is configured to couple with an irrigation liquid source and to pass liquid into said catheter.

11. The device of claim 7, further wherein said second end portion of said aspiration catheter includes one or more openings such that suction for aspiration is created at said second end portion when said first end portion of said aspiration catheter is coupled to the suction source.

12. A method for removing an obstruction from a medical drainage tube, wherein said method comprises:
inserting a device into a lumen of said medical drainage tube, wherein said device comprises:
a catheter with a proximal end portion and a distal tip portion, said distal tip portion including an aperture;
an end cap fixedly coupled to said proximal end portion of said catheter;
an aspiration catheter with a first end portion and a second end portion, said aspiration catheter being generally coaxial with said catheter, wherein said first end portion is arranged to be coupled to a suction source and manipulated by an operator of said device; and
an expandable member at said second end portion of said aspiration catheter, wherein said expandable member is arranged to expand from a low-profile configuration to an enlarged configuration;
passing said distal tip through said obstruction;
causing said expandable member to emerge out from inside said catheter and through said aperture, whereby said expandable member reconfigures from said low-profile configuration to said enlarged configuration; and
retracting said device from said medical drainage tube such that said expandable member contacts said obstruction to move said obstruction out of said medical drainage tube.

13. The method of claim 12, further comprising injecting an irrigation liquid into said catheter.

14. The method of claim 13, further comprising aspirating said irrigation liquid through one or more openings in said second end portion of said aspiration catheter.
